(19) United States
(12) Patent Application Publication
(10) Pub. No.: US 2019/0353644 A1
(43) Pub. Date: Nov. 21, 2019

(54) PROFILE ANALYSIS OF CELL POLARITY

(71) Applicants: Wake Forest University Health Sciences, Winston-Salem, NC (US); Wake Forest University, Winston-Salem, NC (US)

(72) Inventors: Pierre-Alexandre Vidi, Winston-Salem, NC (US); Keith Bonin, Winston-Salem, NC (US)

(21) Appl. No.: 16/415,747

(22) Filed: May 17, 2019

Related U.S. Application Data
(60) Provisional application No. 62/672,951, filed on May 17, 2018.

(51) Int. Cl. G01N 33/50; G01N 33/574; G01N 33/58; G06K 9/00; G06T 7/00

(52) U.S. Cl. CPC G01N 33/5091; G01N 33/57415; G01N 33/582; G06K 9/0014; G06T 7/0012; G06T 2207/30024; G06T 2207/30068; G06T 2207/10056; G06T 2207/10064; G06T 2207/20021; G01N 2800/50

(57) ABSTRACT

The invention relates to methods for quantitating cell (e.g., epithelial cell) apical-basal polarity in three-dimensional multi-cell structures. The invention further relates to methods of using quantitation assays to screen for agents that modulate polarity, assess the risk of developing cancer, and monitor the effectiveness of cancer prevention programs.

### CROSS-REFERENCE TO RELATED APPLICATIONS

This application claims the benefit of U.S. Provisional Application No. 62/672,951, filed May 17, 2018, the entire contents of which are incorporated by reference herein in its entirety.

### FIELD OF THE INVENTION

The invention relates to methods for quantitating cell (e.g., epithelial cell) apical-basal polarity in three-dimensional multi-cell structures. The invention further relates to methods of using quantitation assays to screen for agents that modulate polarity, assess the risk of developing cancer, and monitor the effectiveness of cancer prevention programs.

### BACKGROUND OF THE INVENTION

Preventing cancer is the ultimate goal to reduce the burden of the disease. In order to screen for drugs or evaluate clinical interventions aimed at lowering cancer risk, quantitative readouts of cancer initiation are needed. While many screening platforms have been developed around cancer models to identify new cancer treatments, the equivalent with normal cells for prevention is missing. The following problems can in part explain this current imbalance: (1) cancer initiation mechanisms are generally poorly understood and (2) in vitro cell models of normal cells and tissue are more challenging than tumor models.

In the breast and other epithelia, apical-basal polarity is central to homeostasis and is one of the first tissue characteristics lost during cancer initiation (Lelievre, J. Mammary Gland Biol. Neoplasia 15(1):49 (2010); Royer et al., Cell Death Differ. 18(9):1470 (2011); Martin-Belmonte et al., Nature Rev. Cancer 2012;12(1):23 (2012)).

The mammary gland consists of an arborescence of ducts connecting the glandular elements (called acini, lobules, or alveoli) to the nipple (FIGS. 1A-1B). Several (5-10) of these ductal systems are typically present in each breast (Love et al., Cancer 101:1947 (2004)). The mammary gland is a simple epithelial tissue composed of a single layer of luminal cells lining the ducts and acini (FIG. 1C). Luminal cells are surrounded by myoepithelial cells with contractile function to expel the milk towards the nipple. Myoepithelial cells also secrete most of the factors constituting the basement membrane (BM), a specialized form of extracellular matrix (ECM) lining the epithelium and rich in collagen type IV and laminins. In mammary ducts and acini, apical-basal polarity structurally and functionally defines the cellular organization relative to the lumen and BM (Roignot et al., Cold Spring Harbor Perspectives Biol. 5 (2013); Rodriguez-Boulan et al., Nature Rev. Mol. Cell Biol. 15:225 (2014)). Apical membranes of luminal cells delineate the luminal space and are segregated from basolateral membranes by cell-cell junctions; these different junctional complexes occupy distinct radial positions along the apical-basal polarity axis of the epithelial layer (FIG. 1D).

Tight junctions (TJs) are localized closest to the lumen. They consist of integral membrane proteins (claudins, occludin, JAM (Krug et al., Semin. Cell Dev. Biol. 36:166 (2014))), as well as cytosolic adaptor and scaffolding factors (zona occludens proteins ZO-1, ZO-2, ZO-3 (Gonzalez-Mariscal et al., Semin. Cell Dev. Biol. 11:315 (2000))) bridging the membrane-integral TJ factors with the cytoskeleton. TJs form a seal ensuring the segregation of apical and basolateral membrane lipids and proteins. In addition to this fence function, TJs serve as gates for selective diffusion between basal and luminal interstitial spaces. Both gate and fence functions are essential for the normal function of the gland, in particular for milk secretion and to control paracellular exchanges between blood and milk (Stelwagen et al., J. Mammary Gland Biol. Neoplasia. 2014; 19:131 (2014)).

Adherens junctions (AJs) are located next to TJs and are composed of transmembrane cadherins and nectins bound to cytosolic catenins and to afadin. AJs provide attachment of neighboring cells and are physically bound to TJs via ZO-1. During cell differentiation, AJ formation precedes and promotes TJ assembly by nucleating TJ proteins ( Martin-Belmonte et al., Nature Rev. Cancer 12:23 (2012); Campbell et al., Exp. Cell Res. 358:39 (2017)). Both TJs and AJs are connected to the actin cytoskeleton, with ZO proteins and catenins directly binding to and organizing F-actin, which leads to the establishment and maintenance of perijunctional actomyosin rings stabilizing junctional complexes ( Van Itallie et al., Mol. Biol. Cell 20:3930 (2009)); Arnold et al., Exp. Cell Res. 358:20 (2017)).

Desmosomes have a similar organization as AJs but, in contrast to AJs that are linked to actin filaments, desmosomes are connected to keratin intermediate filaments. Desmosomes also play an important role in cell-cell adhesion along the basolateral membrane. Together with AJs, desmosomes mechanically couple neighboring epithelial cells, and thereby provide mechanical strength to the tissue, define cell-intrinsic mechanical properties, and constitute mechanotransduction hubs for the integration of physical cues from surrounding cells (Broussard et al., Cell Tissue Res. 360:501 (2015); Rubsam et al., Cold Spring Harbor Perspectives Biol. 10 (2018)).

Cell-cell contacts in the breast epithelium and other epithelia also comprise gap junctions (GJs) that form channels connecting the cytoplasm of adjacent cells and that enable cell-cell communication via small molecules (Bazzoun et al., Pharmacol. Therap. 138:418 (2013)). GJs consist of connexons (connexin hexamers) and are classically represented towards the basal side of epithelial cells. Yet connexin 43 was recently found to be apically localized in the breast epithelium, and to be required for apical polarity establishment and maintenance (Adissu et al., J. Cell Sci. in press).

Three major polarity complexes regulate the maturation and maintenance of cell-cell adhesion complexes along the apical-basal axis (reviewed in Roignot et al., Cold Spring Harbor Perspectives Biol. 5 (2013); Martin-Belmonte et al., Nature Rev. Cancer 12:23 (2012)): the crumbs complex, which defines apical membrane identity, the PAR (partitioning defective) system, which defines the apical-basal boundary, and the scribble complex, which defines basolateral membrane identity. The establishment of the apical-basal polarity axis—and particularly, the orientation of this axis orthogonal to the BM—also depends on cell-ECM interactions, which are critical for differentiation and homeostasis (Barcellos-Hoff et al., Development 1989; 105:223 (1989); Bissell et al., Cancer Res. 1999; 59:1757s). Such cell-ECM contacts involve both luminal and myoepithelial cells and are largely mediated by integrins located at the basal pole of the acini and ducts. Integrins cross-talk with and modulate growth factor receptors signaling, and play important roles in mechanosensing (Wang et al., Proc. Natl. Acad. Sci. USA 95:14821 (1998); Glukhova et al., Curr. Opin. Cell Biol. 25:633 (2013); Bosch-Fortea et al., Curr. Opin. Cell Biol. 50:42 (2018)). Importantly, these ECM receptors initiate a structural continuum between ECM and cell nucleus, which defines nuclear shape and genomic functions (Simon et al., Nature Rev. Mol. Cell Biol. 12:695 (2011)).

The function and relevance of cell-cell junctional complexes and cell-ECM contacts go far beyond their structural role. Polarity factors include tumor suppressors and oncoproteins that localize both at cell-cell junctions and in the cytosol or cell nucleus where they modulate biochemical signals, gene expression, and genome maintenance (Balda et al., EMBO J. 19:2024 (2000); Feigin et al., Cancer Res. 74:3180 (2014); Fang et al., Cell Res. 17:100 (2007)). Altered cell polarity causes misregulation of proliferative and survival pathways by shifting the proportion of soluble and membrane bound polarity factors. The inventors also found evidence that cell-ECM interactions are required for an efficient DNA damage response in breast epithelial cells (Vidi et al., J. Cell Sci. 125:350 (2012)). Apical-basal polarity, specifically the PAR system, also defines the orientation of mitotic spindle poles, and hence the relative position of the daughter cells after cytokinesis; spindle orientation parallel to the BM is necessary for the maintenance of a single cell layer and, accordingly, epithelial polarity loss may promote cell multilayering and hyperplasia (McCaffrey et al., Trends Cell Biol. 21:727 (2011); Macara et al., Curr. Biol. 24:R815 (2014)). Epithelial polarity may therefore be considered an architectural biomarker of breast cancer risk and, indeed, disruption of epithelial polarity is one of the first identifiable events and a necessary step for the initiation of carcinoma (Martin-Belmonte et al., Nature Rev. Cancer 12:23 (2012); Lelievre, S A. J. Mammary Gland Biol. Neoplasia. 15:49 (2010); Royer et al., Cell Death Differ. 18:1470 (2011); Chatterjee et al., Breast Cancer 6:15 (2014)).

Current breast cancer risk assessment methods such as the Gail model (Gail et al., J. Natl. Cancer Inst. 81:1879 (1989)) provide population-based estimates of risk. Several genetic breast cancer risk factors have been identified (BRCA1, BRCA2, p53, etc.), yet the majority of breast cancers still have no clear germline mutation origin and cannot be predicted by genetic testing. Molecular assays of breast cancer risk are therefore needed for primary breast cancer prevention research and, ultimately, for personalized cancer prevention.

### SUMMARY OF THE INVENTION

The present invention is based on the concept that breast epithelial polarity, which is a hallmark of homeostasis in the mammary gland, is one of the molecular links between metabolic risk factors (including obesity and prediabetes) and cancer initiation. As such, epithelial polarity readouts may provide valid estimates of cancer risk. Loss of epithelial polarity, and in particular TJ and AJ remodeling, is associated with cancer initiation in multiple contexts, often involving tissue inflammation. For example, ulcerative colitis and Crohn's disease are both associated with elevated colorectal cancer risk (Dulai et al., Cancer Prevention Res. 9:887 (2016)) and are characterized by TJ dysfunctions (Vancamelbeke et al., Expert Rev. Gastroenterol. Hepatol. 11:821 (2017)). Similarly, patients with Celiac disease have TJ defects and increased epithelial cell permeability in the small intestine. These patients are at increased risk for intestinal lymphoma and small bowel cancer. For breast cancer, obesity is one of the few modifiable risk factors and is characterized by a chronic state of inflammation and deregulation of cytokine and growth factors in circulation (Khandekar et al., Nature Rev. Cancer 11:886 (2011); Dietze et al., Am. J. Pathol. 188:280 (2018)). The inventors found that cell microenvironments characteristic of obesity lead to the mislocalization of apical polarity proteins and premalignant changes in the mammary gland (Adissu et al., J. Cell Sci. in press; Tenvooren et al., Oncogene (2019)). Apical polarity was also found to be disrupted by omega-6 fatty acids, which may be associated with increased breast cancer risk (Yue et al., Biophys. J. 102:1215 (2012)). These observations validate the concept of using epithelial polarity as readout for primary prevention.

Apical-basal polarity has not been used before as a readout for cancer prevention, e.g., in screening assays. Several methods have been described to quantify polarity in epithelial cells, including (1) visual scoring of polarity markers by an investigator, using an epifluorescence microscope; (2) measuring paracellular flux with dextrans (or other particles), typically in transwell assays; (3) transepithelial electrical resistance measurements; and (4) Raman microscopy (analysis of apical vs. basolateral cell membrane characteristics). In their current forms, however, these approaches are not compatible with automated, high-throughput analyses required for drug screening or diagnostics.

The present invention overcomes the deficiencies in the art by providing methods and devices for quantitating cell (e.g., epithelial cell) polarity based on apical-basal polarity marker distributions and approaches to evaluating cancer risk based on the polarity quantitation.

The invention is based on the evaluation of polarity marker distribution and essentially replaces the human investigator scoring at the microscope by an automated image analysis procedure based on profile analyses, e.g., radial profile (RP) analyses, of polarity marker distributions. The end result is an objective readout of epithelial cell polarity, i.e., a polarity score. A higher polarity score indicates a higher amount of polarity in cells and a lower polarity score indicates a lower amount of polarity in cells (e.g., a loss of polarity). Each step of the approach illustrated in the flow chart (FIG. 2) is amenable to automation.

Accordingly, one aspect of the invention relates to a method for quantitating epithelial cell apical-basal polarity in a three-dimensional multi-cell structure, comprising:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating epithelial cell polarity.

Another aspect of the invention relates to a method of identifying an agent that modulates epithelial cell apical-basal polarity, comprising quantitating epithelial cell apical-basal polarity in a three-dimensional multi-cell structure in the presence and absence of an agent,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating epithelial cell polarity;
wherein an increase in the polarity score in the presence of the agent compared to the absence of the agent identifies the agent as an agent that increases epithelial cell apical-basal polarity, and
a decrease in the polarity score in the presence of the agent compared to the absence of the agent identifies the agent as an agent that decreases epithelial cell apical-basal polarity.

A further aspect of the invention relates to a method of identifying an agent that may be effective for chemoprevention of epithelial cancer, comprising quantitating epithelial cell apical-basal polarity in a three-dimensional multi-cell structure in the presence and absence of an agent,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating epithelial cell polarity;
wherein an agent that inhibits a decrease in epithelial cell apical-basal polarity or increases epithelial cell apical-basal polarity is identified as an agent that may be effective for chemoprevention of epithelial cancer.

An additional aspect of the invention relates to a method for assessing the risk of developing epithelial cancer in a subject, comprising:
obtaining or having obtained a blood sample from the subject;
contacting a three-dimensional multi-cell structure with the blood sample; and
quantitating epithelial cell apical-basal polarity in the three-dimensional multi-cell structure in the presence and absence of the blood sample,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating epithelial cell polarity;
wherein a decrease in the polarity score in the presence of the blood sample compared to a control (e.g., the absence of the blood sample or a baseline blood sample) indicates an increased risk of developing epithelial cancer.

Another aspect of the invention relates to a method for assessing the risk of developing epithelial cancer in a subject, comprising:
obtaining or having obtained a tissue sample comprising epithelial cells from the subject; quantitating epithelial cell apical-basal polarity in a three-dimensional multi-cell structure in the tissue sample,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating epithelial cell polarity;
wherein a lower polarity score compared to the polarity score in a healthy population indicates an increased risk of developing epithelial cancer.

A further aspect of the invention relates to a method for monitoring for a change in the risk of developing epithelial cancer in a subject, comprising:
obtaining or having obtained two or more blood samples from the subject over time;
contacting a three-dimensional multi-cell structure with each blood sample; and
quantitating epithelial cell apical-basal polarity in the three-dimensional multi-cell structure in the presence of each blood sample,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating epithelial cell polarity;
wherein an increase in the polarity score over time indicates a decrease in the risk of developing epithelial cancer, and
a decrease in the polarity score over time indicates an increase in the risk of developing epithelial cancer.

An additional aspect of the invention relates to a method for monitoring for a change in the risk of developing epithelial cancer in a subject, comprising:
obtaining or having obtained two or more tissue samples comprising epithelial cells from the subject over time;
quantitating epithelial cell apical-basal polarity in a three-dimensional multi-cell structure in each of the tissue samples,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating epithelial cell polarity;
wherein an increase in the polarity score over time indicates a decrease in the risk of developing epithelial cancer, and
a decrease in the polarity score over time indicates an increase in the risk of developing epithelial cancer.

Another aspect of the invention relates to a method for monitoring the effectiveness of a cancer prevention program, comprising:
obtaining or having obtained at least one blood sample from the subject before starting the cancer prevention program and at least one blood sample from the subject during and/or after the cancer prevention program;
contacting a three-dimensional multi-cell structure with each blood sample; and
quantitating epithelial cell apical-basal polarity in the three-dimensional multi-cell structure in the presence of each blood sample,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating epithelial cell polarity;
wherein an increase in the polarity score or a lack of decrease in the polarity score over time indicates the cancer prevention program is effective, and
a decrease in the polarity score over time indicates the cancer prevention program is not effective.

A further aspect of the invention relates to an electronic device comprising:
a processor; and
a storage medium coupled to the processor and comprising computer readable program code therein that when executed by the processor causes the processor (350) to perform the methods of the invention.

An additional aspect of the invention relates to a computer program product comprising:
a non-transitory computer readable storage medium comprising computer readable program code therein that when executed by a processor causes the processor to perform the methods of the invention.

Another aspect of the invention relates to an electronic device comprising: a processor; and
a storage medium coupled to the processor and comprising computer readable program code that when executed by the processor causes the processor to perform operations comprising:
a) capturing an image of three-dimensional multi-cell structure in which a polarity marker has been detected;
b) segmenting the image into multicellular units having a level of apical-basal polarity;
c) measuring intensity of the polarity marker across a multicellular unit; and
d) calculating a polarity score for the multicellular unit based on the intensity distribution of the polarity marker.

A further aspect of the invention relates to a computer program product comprising: a non-transitory computer readable storage medium comprising computer readable program code therein that when executed by a processor causes the processor to perform operations comprising:
a) capturing an image of three-dimensional multi-cell structure in which a polarity marker has been detected;
b) segmenting the image into multicellular units having a level of apical-basal polarity;
c) measuring intensity of the polarity marker across a multicellular unit; and
d) calculating a polarity score for the multicellular unit based on the intensity distribution of the polarity marker.

An additional aspect of the invention relates to a kit comprising a computer program product of the invention.

These and other aspects of the invention are set forth in more detail in the description of the invention below.

### BRIEF DESCRIPTION OF THE DRAWINGS

FIGS. 1-1F show apical-basal polarity in the normal mammary gland and in culture models of acini. (A) Schematic of the breast anatomy. Different ductal systems (or lobes) are shown in distinct shades. TDLU, terminal ductal lobular unit (the initiation site for most breast carcinomas). (B) Immunohistochemistry (IHC, bottom) and immunofluorescence (IF, top) images of normal breast tissue sections. (C) Schematic and higher magnification images of functional glandular units (acini) stained with the ZO-1 and β4-integrin epithelial polarity markers. (D) Schematic of cell junctional complexes along the apical-basal polarity axis of the epithelium, and functional effects of epithelial polarity loss in cancer initiation. BM, basement membrane; DC, desmocollin; DG, desmoglein; DP, desmoplakin; IF, intermediate filaments. (E) Schematic and representative confocal images of a breast acinus produced in 3D culture. The IHC image in B is from the Komen Tissue Bank. Scale bars, 200 μm (B) and 20 μm (C and E). (F) Immunostaining for the structural nuclear protein NuMA in a 2D monolayer culture (top), in a 3D culture of acini (middle), and in normal human breast tissue (bottom). Orthogonal views of stained nuclei (DAPI) in 2D and 3D cultures are shown on the right.

FIG. 2 shows a flowchart of operations at an electronic device for quantitating cell polarity.

FIG. 3 shows an example of a RadialProfiler flowchart. (1) Images are taken from acini cultures stained with a nuclear dye (2) and for a cell polarity marker (3). (4) Acini are segmented based on the DNA dye. Filtering steps exclude structures with inappropriate sizes or structures that are out of focus. (5) Acini are divided into concentric terraces used to calculate radial profiles of polarity. (6) The profiles are normalized and integrated to obtain a summary value of polarity (RP index). Scale bar, 50 μm. See text for details.

FIGS. 4A-4C show elimination of out-of-focus acini. (A) Illustration of the wavelets (WAVR) blur metric calculated for Hoechst images with different levels of Gaussian blur. (B) Representative acini images (Hoechst stain) deemed either in focus or out of focus and their corresponding WAVR values. Images in A and B were taken with an epifluorescence microscope at 20× magnification, using a sCMOS camera. (C) Histograms showing the probability density function of WAVR values belonging to acini images visually rated as clear (in focus; n=76) or blurry (out of focus; n=39). Corresponding Levenberg-Marquardt fits of normal distributions are shown in blue circles and red circles for the two distributions.

FIGS. 5A-5B show graphical user interfaces of RadialProfiler. (A) Window to select image folders corresponding to the dataset for analysis, and to define analysis parameters. The user chooses between supervised and unsupervised analyses with this first dialog box. (B) Interface assisting visual scoring of polarity marker distribution. This window appears when the user selects supervised analysis. For each acinus identified by RadialProfiler (in the entire dataset selected in A), nuclear stain and polarity images are displayed side-by-side. The user input is a binary choice between (‘Polar’ or ‘Non-Polar’) or exclusion from analysis. The progress bar (bottom) indicates the number of structures that remain to be scored. Acini appear in a randomized order.

FIGS. 6A-6B show RadialProfiler analysis of wide field fluorescence images from fixed and immunostained acini (A), or of cortical actin staining in live acini (B). The figure shows (1) portions of overlay images, (2) nuclear stain images (inverted to improve visualization), (3) corresponding masks with the concentric terraces, (4) inverted polarity images, and (5) polarity images annotated with acini contours and RP indexes. In rare instances (arrowhead in A-4), acini were under-segmented. In B, an overlay of the bright field image and the corresponding contour ROI validates the segmentation (6). Scale bars, 100 μm (A) and 20 μm (B).

FIGS. 7A-7C show illustration of RadialProfiler results for HMT-3522 S1 acini in different culture vessels. The supervised version of the software was used to classify acini in polarized and nonpolarized categories. Radial profiles (left) and bar graphs of the RP indexes (right) are shown for both categories. (A) Fixed acini immunostained for ZO-1. (B-C) Live imaging of acini stained with SiR-actin. Fluorescence images were captured using a wide field microscope (Olympus; A and B) or with an automated spinning disc high content imaging system (Perkin Elmer Operetta; C). In C, maximal intensity projections of 10 confocal frames were analyzed. The number of radial bins used for analysis was adapted to the different magnifications and image resolutions. ****, P<0.0001 (Student's t-test).

FIGS. 8A-8G show computational image analysis of apical polarity. (A) Fluorescence microscopy images of differentiated acini stained for the tight junction marker ZO-1. Nuclei were counterstained with DAPI. Scale bar, 100 μm. (B) Evaluation of two computational approaches for the quantification of ZO-1 signals, based on radial profiles of signal intensities (RP, N=10 bins), or on moments of inertia (MOI). For both approaches, a set of acini images was visually selected for different levels of apical polarization (illustrations on the left). (C) Quantification of the radial distribution of ZO-1 staining signals in acini treated for 72 h with vehicle or with leptin (100 ng/ml). The curves and integrated values represent averages from eight independent experiments. ***, P<0.0001 (unpaired t-test; 150-450 acini were analyzed for each replicate). (D) Visual scoring of ZO-1 distribution in acini treated with vehicle, with leptin, or with leptin and a leptin receptor antagonist (LA). Representative confocal images are shown. *, P<0.05 (ANOVA and Tukey's post-hoc test). (E) The effect of elevated estrogen on polarity of breast acini. (F) The effect of lipopolysaccharide on polarity of breast acini. (G) The effect of transient exposure to elevated growth factors on polarity of breast acini.

FIGS. 9A-9B show images of mammary epithelial cells (HMT-3522 S1) expressing GFP-ZO-1. (A) 2D cell culture and (B) 3D culture.

FIG. 10A is a block diagram of an electronic device that is configured to quantitate cell polarity, according to embodiments of the present inventive concepts.

FIG. 10B is a block diagram that illustrates details of an example processor and memory that may be used in accordance with various embodiments.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

### Definitions

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or “decrease” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

As used herein, the terms “protein” and “polypeptide” are used interchangeably and encompass both peptides and proteins, unless indicated otherwise.

A “fusion protein” is a polypeptide produced when two heterologous nucleotide sequences or fragments thereof coding for two (or more) different polypeptides not found fused together in nature are fused together in the correct translational reading frame. Illustrative fusion polypeptides include fusions of a polypeptide of the invention (or a fragment thereof) to all or a portion of glutathione-S-transferase, maltose-binding protein, or a reporter protein (e.g., green fluorescent protein, β-glucuronidase, β-galactosidase, luciferase, etc.), hemagglutinin, c-myc, FLAG epitope, etc.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide or fragment can also function in intact cells without purification.

### Methods for Quantitating Cell Polarity

The present invention provides methods for quantitating epithelial cell polarity based on apical-basal polarity marker distributions.

Accordingly, one aspect of the invention relates to a method for quantitating cell (e.g., epithelial cell) apical-basal polarity in a three-dimensional multi-cell structure, comprising:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating cell (e.g., epithelial cell) polarity.

The polarity to be detected in the three-dimensional multi-cell structure may be of any pattern found in epithelial cell structures. In some embodiments, the polarity of the multicellular unit may be radial polarity, e.g., where the epithelial cells form or surround a lumen. In other embodiments, the polarity may be angular (e.g., azimuthal).

The three-dimensional multi-cell structure may be any structure in which epithelial cells are arranged in a manner that exhibits a polarity pattern. In some embodiments, the three-dimensional multi-cell structure is a naturally occurring structure, e.g., a structure present in a tissue sample. In some embodiments, the structure may be a slice of a tissue sample having a thickness of about 6-10 μm, e.g., about 8 μm. In some embodiments, the three-dimensional multi-cell structure is a three dimensional cell culture comprising epithelial cells (e.g., an organoid culture), wherein the cells form a structure having a polarity pattern (i.e., a “cell culture equivalent” of a naturally occurring structure). The cell culture structures may be in individual wells of a multiwell plate, with each well containing one or more than one structure. 3D cultures are generally performed by placing cells on a hydrogel or equivalent substrate and providing the proper physical and chemical cues to the cells for differentiation. Cells may be cultured inside or on top of these matrices (see, e.g., Vidi et al., Methods Mol. Biol. 945:193 (2013), incorporated by reference herein in its entirety).

In certain embodiments, when the three-dimensional multi-cell structure is a naturally occurring structure it may comprise at least a portion of an exocrine gland (acinus), e.g., at least one intact exocrine gland. When the three-dimensional multi-cell structure is a three dimensional cell culture it may comprise a cell culture equivalent of an exocrine gland (e.g., where the cultured cells form an acinus-like structure).

The three-dimensional multi-cell structure may comprise cells from a tissue comprising exocrine glands or from an epithelial cell line derived from a tissue comprising exocrine glands. For example, the tissue comprising exocrine glands may be breast, liver, pancreas, or any other tissue known to comprise exocrine glands.

In certain embodiments, the three-dimensional multi-cell structure may comprise epithelial structures other than an exocrine gland, e.g., a linear structure or other non-globular structure.

In the method of the invention, at least one polarity marker is detected in the three-dimensional multi-cell structure in order to determine a level of apical-basal polarity in the multicellular unit. The level of polarity may range from 0% (no polarity) to 100% (complete polarity and any number in-between). In some embodiments, two or more polarity markers are detected in the three-dimensional multi-cell structure, e.g., 2, 3, 4, 5, or 6 or more. In certain embodiments, when two or more polarity markers are detected, they may be present in different parts of the cell, e.g., one may be an apical marker and one may be a basal marker. In some embodiments, each polarity marker is detected using a different detection method or a method that produces distinguishable signals.

The polarity marker may be any molecule or structure that is present in an epithelial cell in a manner that indicates the polarity of the cell (i.e., present in a polarized manner). The polarity marker may be a protein, lipid, organelle, or other molecule or structure that is present in a polarized manner.

In some embodiments, the polarity marker is a protein that is expressed in a polarized manner. In some embodiments, the protein is predominantly expressed or present on the apical side of the cell. The term “predominantly,” as used herein, refers to a polarity marker that is unequally distributed in a cell to a sufficient amount that it indicates the polarity of the cell. Examples of apical polarity markers include, without limitation, proteins that are expressed in a cell-cell junction, such as a tight junction, adherens junction, or gap junction. In some embodiments, the protein is predominantly expressed on the basal side of the cell. Examples of basal polarity markers include, without limitation, proteins that are involved in cell attachment to the extracellular matrix or secreted factors located in the extracellular matrix. Examples of protein polarity markers include, without limitation, afadin, claudin-1, ZO-1, ZO-2, ZO-3, Par3, a connexin, E-cadherin, β-catenin, GM130, β4-integrin, α6-integrin, or collagen IV.

In some embodiments, the polarity marker may be a cytoskeleton element that is present in the cell in a polarized manner. An example, incudes, without limitation, cortical actin filament.

In some embodiments, the polarity marker may be an intracellular molecule such as a phosphoinositide.

In some embodiments, the polarity marker may be an organelle such as a basally-localized cell nucleus, apical-oriented Golgi bodies or vacuoles, etc.

The polarity marker may be detected by any method known in the art to be suitable for detecting the subcellular location of a marker. In some embodiments, the polarity marker may be detected using a binding assay using a reagent that specifically binds the polarity marker and is itself visually detectable. Examples of reagents include, without limitation, an antibody, an affinity agent (such as an aptamer or labeled peptide), or a dye (e.g., SiR-actin, which binds cortical actin filaments). In some embodiments, the reagent itself may be detectable with visible light, fluorescent light, or another light source. In other embodiments, the reagent is an enzyme that is detectable through enzymatic conversion of a substrate to a detectable molecule (e.g., horseradish peroxidase). When the polarity marker is a protein, it may be fused with an amino acid sequence that is a detectable marker and the fusion protein may be introduced into epithelial cells, e.g., in culture. The fusion protein can then be detected in the three dimensional structure based on the detectable marker portion of the fusion protein, e.g., by providing an antibody that binds the detectable marker (such as a c-myc tag, HA tag, or FLAG epitope) or an enzymatic substrate for the detectable marker (such as β-glucuronidase, β-galactosidase, luciferase, etc.) or a fluorescent protein (such as green fluorescent protein, red fluorescent protein, etc.).

In certain embodiments, step (a) may further comprise detecting other portions of the cell, e.g., to help identify the individual cells and the overall structure of the three-dimensional multi-cell structure. For example, the nucleus may be labeled using a DNA stain such as DAPI and/or cell membranes may be labeled using a membrane stain.

Following the detection of the polarity marker, an image (e.g., a microscope image) of the three-dimensional multi-cell structure is captured. The image may be capture using any suitable device known in the art. Suitable devices include, without limitation, a microscope, camera, digital camera, charge coupled device (CCD), complementary metal-oxide semiconductor (CMOS), etc. In some embodiments, two or more microscope images of the labeled three-dimensional multi-cell structure are captured, e.g., 2, 3, 4, 5, 6, 7, 8, 9, or 10 or more images.

Preferably, each image captures a relevant portion of the three dimensional multi-cell structure, e.g., a portion containing a polarized epithelial structure. In some embodiments, the image captures a single exocrine gland (acinus) or a cell culture equivalent of an exocrine gland.

Following the image capture, the image is processed and analyzed to detect the distribution of the detected polarity marker. The processing incudes identifying the multicellular units within the image that have or may have a polarity pattern (e.g., exocrine glands or cell culture equivalents). The identifying may comprise, for example, identifying the centroid of the multicellular unit and/or the boundary of the multicellular unit. When the multicellular unit is circular (e.g., an acinus), the center of the unit may be identified. The workflow of the image processing may include, for example, the steps of image segmentation, filtering (e.g., to exclude structures that are out of focus, e.g., using Otsu filtering), contour terracing, and polarity score (also called RP index) calculation.

As part of the processing, the intensity of the polarity marker (e.g., the amount of signal produced based on the detection of the marker) across the multicellular unit is measured. This may comprise dividing the area of the multicellular unit into 2 or more bins based on distance from a point in the image and measuring the intensity of the polarity marker in each bin. For example, the multicellular unit may be divided into 5, 10, 15, 20, 25, 30, 35, 40, 45, or 50 or more bins. In some embodiments, the bins are based on the distance calculated from the center of mass of the multicellular unit. In some embodiments, the bins have equal areas.

A polarity score may be calculated for each multicellular unit, e.g., based on the intensity of the polarity marker in each bin. The polarity score is an indicator of the level of polarity in the epithelial cells in the multicellular unit. In some embodiments, the polarity score is a positive number if apical polarity (signal at center of the multicellular unit) is detected and a negative number if basal polarity (peripheral signal) is detected. In some embodiments, the intensity in each bin is normalized to the average intensity in the multicellular unit. The normalized intensity may be plotted versus distance from the point in the image to generate a polarity curve. The polarity curve may be integrated to provide a single value for the polarity score. The polarity score may be used to indicate that status of the cells, e.g., whether the normal polarity pattern of the cells is intact or whether the polarity pattern has been partially or completely disrupted.

In certain embodiments, a polarity score is calculated for at least two multicellular units (e.g., at least 5, 10, 15, 20, 25, 50, 100, 150, 200, or more) and an average polarity score is determined. The at least two multicellular units may be in the same image or different images.

### Applications Based on Quantitating Cell Polarity

As stated above, impaired polarity leads to perturbation of cell homeostasis (e.g., through altered gene expression control) and to cell multilayering and expansion of progenitor cells, both of which are primordia of cancer (Martin-Belmonte et al., Cell 128(2):383 (2007)). Apical-basal polarity can therefore be considered an “architectural” readout of risk (Lee et al., J. Cell Sci. 121(Pt 8):1141 (2008)). The advancements provided by the present invention in detecting and quantitating cell (e.g., epithelial cell) polarity provide numerous applications related to identifying agents that alter cell polarity, determining cancer risk in a subject, and monitoring the effectiveness of cancer prevention programs based on the detection of changes in polarity.

Thus, one aspect of the invention relates to a method of identifying an agent that modulates cell (e.g., epithelial cell) apical-basal polarity, comprising quantitating cell apical-basal polarity in a three-dimensional multi-cell structure in the presence and absence of an agent,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having apical-basal polarity;
d) measuring intensity of the polarity marker across a multicellular unit; and
e) calculating a polarity score for the multicellular unit based on the intensity distribution of the polarity marker;
thereby quantitating cell (e.g., epithelial cell) polarity;
wherein an increase in the polarity score in the presence of the agent compared to the absence of the agent identifies the agent as an agent that increases cell apical-basal polarity, and a decrease in the polarity score in the presence of the agent compared to the absence of the agent identifies the agent as an agent that decreases cell apical-basal polarity.

The three-dimensional multi-cell structure may be either a tissue sample or a cell culture structure. The method may further comprise contacting the structure with a compound or molecule that is known to disrupt apical-basal polarity (a polarity-reducing agent) in order to measure the ability of the agent being screened to reverse the depolarization. Examples of compounds and molecules that are known to disrupt apical-basal polarity include, without limitation, calcium ions, chelators (such as EGTA), fatty acids (such as ω6), adipokines and growth factors (such as excess levels of estrogens and leptin).

Any agent can be screened according to the present invention. Suitable agents include organic and inorganic molecules. Suitable organic molecules can include but are not limited to small molecules (compounds less than about 1000 Daltons), polypeptides (including enzymes, antibodies, and Fab′ fragments), carbohydrates, lipids, coenzymes, and nucleic acid molecules (including DNA, RNA, and chimerics and analogs thereof) and nucleotides and nucleotide analogs.

Further, the methods of the invention can be practiced to screen a compound library, e.g., a small molecule library, a combinatorial chemical compound library, a polypeptide library, a cDNA library, a library of nucleic acids such as antisense nucleic acids or siRNAs, CRISPR/Cas9-based screens, and the like, or an arrayed collection of compounds such as polypeptide and nucleic acid arrays.

The screening may be carried out using automated steps and high throughput methods known in the art, e.g., using multiwell plates comprising three-dimensional multi-cell structures in each well. In one example, three-dimensional acini cultures in a multiwall plate (e.g., 96, 384, or 1536 well plates) may be imaged with an automated confocal imaging system and analyzed using software, such as the RadialProfiler software disclosed herein. Such methods may be carried out using high content analysis systems.

A further aspect of the invention relates to a method of identifying an agent that may be effective for chemoprevention of cancer (e.g., epithelial cancer), comprising quantitating cell (e.g., epithelial cell) apical-basal polarity in a three-dimensional multi-cell structure in the presence and absence of an agent,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating cell polarity;
wherein an agent that inhibits a decrease in cell apical-basal polarity or increases cell apical-basal polarity is identified as an agent that may be effective for chemoprevention of cancer.

The three-dimensional multi-cell structure may be either a tissue sample or a cell culture structure. The method may further comprise contacting the structure with a compound or molecule that is known to disrupt apical-basal polarity (a polarity-reducing agent) in order to measure the ability of the agent being screened to reverse the depolarization. Examples of compounds and molecules that are known to disrupt apical-basal polarity include, without limitation, those listed above.

Identified agents may be those that inhibit a decrease in polarity induced by a polarity-reducing agent or polarity-reducing conditions. Identified agents may be those that increase polarity. Screening with 3D cell culture assays has the advantage of physiological relevance (by contrast with classic 2D cell cultures on plastic), rapid turnover, manipulability, and lower costs and higher throughput compared to animal models.

Any agent can be screened as described above. The screening may be carried out using automated steps and high throughput methods known in the art, e.g., using multiwall plates comprising three-dimensional multi-cell structures in each well.

An additional aspect of the invention relates to a method for assessing the risk of developing cancer (e.g., epithelial cancer) in a subject, comprising: obtaining or having obtained a blood sample from the subject;
contacting a three-dimensional multi-cell structure with the blood sample; and
quantitating cell (e.g., epithelial cell) apical-basal polarity in the three-dimensional multi-cell structure in the presence and absence of the blood sample,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating cell polarity;
wherein a decrease in the polarity score in the presence of the blood sample compared to the absence of the blood sample indicates an increased risk of developing cancer.

This method is based on the concept that soluble factors (e.g., hormones, growth factors, toxicants, etc.) that affect cell polarity and increase the risk of developing cancer may be circulating in the blood of some subjects. The cancer may be any type of cancer that is associated with cells the exhibit polarity. The soluble factors may be detected by obtaining a blood, plasma, or serum sample from a subject and contacting a three-dimensional multi-cell structure with the sample to observe the effect on polarity. Other relevant body fluids (e.g., nipple aspirate or saliva) may also be used. Identification of a sample that decreases the polarity of multicellular units in the three-dimensional multi-cell structure identifies the subject as one that has an increased risk of developing epithelial cancer.

Another aspect of the invention relates to a method for assessing the risk of developing cancer (e.g., epithelial cancer) in a subject, comprising: obtaining or having obtained a tissue sample comprising cells (e.g., epithelial cells) from the subject;
quantitating cell (e.g., epithelial cell) apical-basal polarity in a three-dimensional multi-cell structure in the tissue sample,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating cell polarity;
wherein a lower polarity score compared to the polarity score in a healthy population indicates an increased risk of developing cancer.

This method is based on measuring the current state of polarity in epithelial cells in a sample from a subject. A finding of lower polarity score in the sample relative to the average polarity score in samples from the general population (e.g., subjects that do not have cancer) is indicative of an increased risk of developing epithelial cancer. In some embodiments, the polarity score in the tissue sample may be compared to the polarity score in a healthy tissue sample from the same subject or from average values from low-risk healthy individuals.

A further aspect of the invention relates to a method for monitoring for a change in the risk of developing cancer (e.g., epithelial cancer) in a subject, comprising: obtaining or having obtained two or more blood samples from the subject over time; contacting a three-dimensional multi-cell structure with each blood sample; and quantitating cell (e.g., epithelial cell) apical-basal polarity in the three-dimensional multi-cell structure in the presence of each blood sample,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating cell polarity;
wherein an increase in the polarity score over time indicates a decrease in the risk of developing cancer, and
a decrease in the polarity score over time indicates an increase in the risk of developing cancer.

An additional aspect of the invention relates to a method for monitoring for a change in the risk of developing cancer (e.g., epithelial cancer) in a subject, comprising: obtaining or having obtained two or more tissue samples comprising cells (e.g., epithelial cells) from the subject over time;
quantitating cell apical-basal polarity in a three-dimensional multi-cell structure in each of the tissue samples,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating cell polarity;
wherein an increase in the polarity score over time indicates a decrease in the risk of developing cancer, and
a decrease in the polarity score over time indicates an increase in the risk of developing cancer.

Another aspect of the invention relates to a method for monitoring the effectiveness of a cancer prevention program, comprising:
obtaining or having obtained at least one blood sample from the subject before starting the cancer prevention program and at least one blood sample from the subject during and/or after the cancer prevention program;
contacting a three-dimensional multi-cell structure with each blood sample; and quantitating cell (e.g., epithelial cell) apical-basal polarity in the three-dimensional multi-cell structure in the presence of each blood sample,
wherein the quantitating comprises:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating cell polarity;
wherein an increase in the polarity score or a lack of decrease in the polarity score over time indicates the cancer prevention program is effective, and
a decrease in the polarity score over time indicates the cancer prevention program is not effective.

The above methods are based on monitoring the state of polarity in epithelial cells in a sample from a subject over time. The monitoring may take place by measuring changes in the blood, plasma, or serum of a subject as determined by effect on polarity a three-dimensional multi-cell structure or by measuring the polarity in epithelial tissue samples from the subject. Samples may be obtained and tested on a regular basis, e.g., monthly, semiannually or annually or every 2, 3, 4, 5, 6, 7, 8, 9, or 10 years. A finding that the polarity score is decreasing over time is an indicator that the risk of developing epithelial cancer is increasing. A finding that the polarity score is steady over time is an indicator that the risk of developing epithelial cancer is unchanged. A finding that the polarity score is increasing over time is an indicator that the risk of developing epithelial cancer is decreasing. In subjects undergoing a cancer prevention program (e.g., a weight-loss, exercise, and/or diet program or chemoprevention (e.g., metformin)), the method may be used to monitor the effectiveness of the program. Samples may be taken at various times, e.g., before the subject starts the program, at intervals during the program, and/or after the program is completed. A determination that the polarity score in samples from a subject participating in the program is increasing, remaining the same, or decreasing at a slower rate than before participation in the program indicates that the program is effective. A determination that the polarity score in samples from a subject participating in the program continues to decrease at a rate similar to the rate before participation in the program indicates that the program has not been effective. This may indicate that the program should be continued for a longer period until effectiveness is observed or that different prevention measures should be considered. Compared to prospective studies to quantify the intervention effect based on cancer occurrence that take decades, the present invention provides a rapid complementary analysis of risk.

In some embodiments, the blood sample or other body fluid or the tissue sample may be obtained from the subject by the person carrying out the entire method of the invention. In other embodiments, the blood sample or other body fluid or the tissue sample may be obtained from the subject by one person (e.g., a physician, nurse, or phlebotomist) and the remainder of the method performed on the sample by a different person (e.g., at a medical laboratory).

In each of the risk assessment or effectiveness determination methods, the methods may further comprise additional steps based on the results of the assay. If the risk of developing epithelial cancer is increased, further steps may include increased frequency and/or intensity of monitoring the subject for the development of cancer. Other steps may include placing the subject on a cancer prevention program or increasing the intensity of the cancer prevention program. Other steps may include treating the patient with appropriate treatments for early stage cancer.

The present invention finds use in veterinary and medical applications. Suitable subjects include avians, reptiles, fish, and mammals, with mammals being preferred. The term “mammal” as used herein includes, but is not limited to, humans, bovines, ovines, caprines, equines, felines, canines, lagomorphs, etc. Human subjects include neonates, infants, juveniles, and adults. In other embodiments, the subject is an animal model of cancer. In certain embodiments, the subject has or is at risk for cancer.

### Products and Kits for Carrying Out the Methods of the Invention

A further aspect of the invention relates to an electronic device comprising: a processor; and
a storage medium coupled to the processor and comprising computer readable program code therein that when executed by the processor causes the processor to perform the methods of the invention.

Another aspect of the invention relates to an electronic device comprising: a processor; and
a storage medium coupled to the processor and comprising computer readable program code that when executed by the processor causes the processor to perform operations comprising:
a) capturing an image of three-dimensional multi-cell structure in which a polarity marker has been detected;
b) segmenting the image into multicellular units having a level of apical-basal polarity;
c) measuring intensity of the polarity marker across a multicellular unit; and
d) calculating a polarity score for the multicellular unit based on the intensity distribution of the polarity marker.

The operations may be performed by an electronic device, which may be a smartphone, a tablet computer, a laptop computer, a portable camera, or one of various other portable electronic devices. Alternatively, the operations may be performed by a server, a desktop computer, a fixed camera, or another electronic device that is separate from, and less portable than, the electronic device. The electronic device may, in some embodiments, be referred to as a “mobile device” or a “user equipment.”

The electronic device may further comprise or be connected to other components for carrying out the methods of the invention, such as an image capture device, a network interface, a microscope, a light source, a graphical user interface, etc. The image capture device may be any camera or other device that captures image data of the three dimensional multi-cell structure that can be used to quantitate polarity.

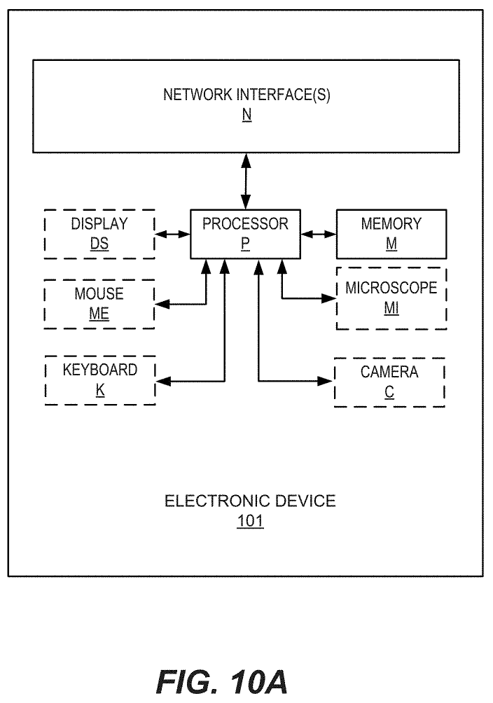

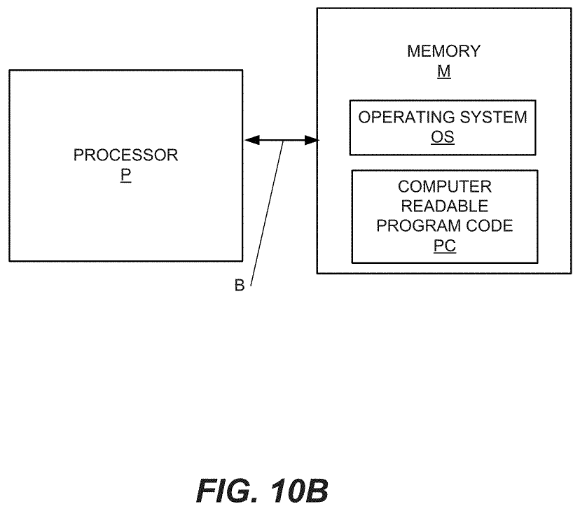

FIG. 10A is a block diagram of an electronic device 101 that is configured to perform image analysis according to embodiments of the present inventive concepts. The electronic device 101 may include a processor P and a memory M. The electronic device 101 may also include network interface(s) N and input/output interface(s), such as a display screen DS, a mouse ME, a keyboard (or keypad) K, a microscope MI, and/or a camera C. The input/output interface(s) may be configured to receive user inputs from a user and/or to display data to the user. In some embodiments, the display screen DS may comprise a touchscreen display.

An additional aspect of the invention relates to a computer program product comprising:
a non-transitory computer readable storage medium comprising computer readable program code therein that when executed by a processor causes the processor to perform the methods of the invention.

A further aspect of the invention relates to a computer program product comprising: a non-transitory computer readable storage medium comprising computer readable program code therein that when executed by a processor causes the processor to perform operations comprising:
a) capturing an image of three-dimensional multi-cell structure in which a polarity marker has been detected;
b) segmenting the image into multicellular units having a level of apical-basal polarity;
c) measuring intensity of the polarity marker across a multicellular unit; and
d) calculating a polarity score for the multicellular unit based on the intensity distribution of the polarity marker.

FIG. 10B is a block diagram that illustrates details of an example processor P and memory M that may be used in accordance with various embodiments. The processor P communicates with the memory M via an address/data bus B. The processor P may be, for example, a commercially available or custom microprocessor. Moreover, the processor P may include multiple processors. The memory M may be a non-transitory computer readable storage medium and may be representative of the overall hierarchy of memory devices containing the software and data used to implement various functions of an electronic device 101 as described herein. The memory M may include, but is not limited to, the following types of devices: cache, ROM, PROM, EPROM, EEPROM, flash, Static RAM (SRAM), and Dynamic RAM (DRAM).

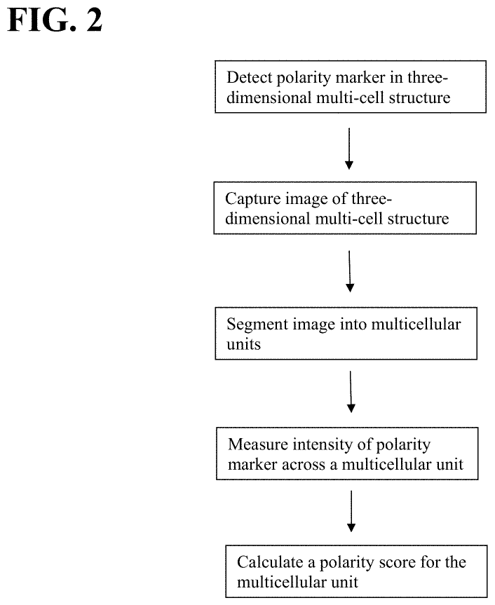

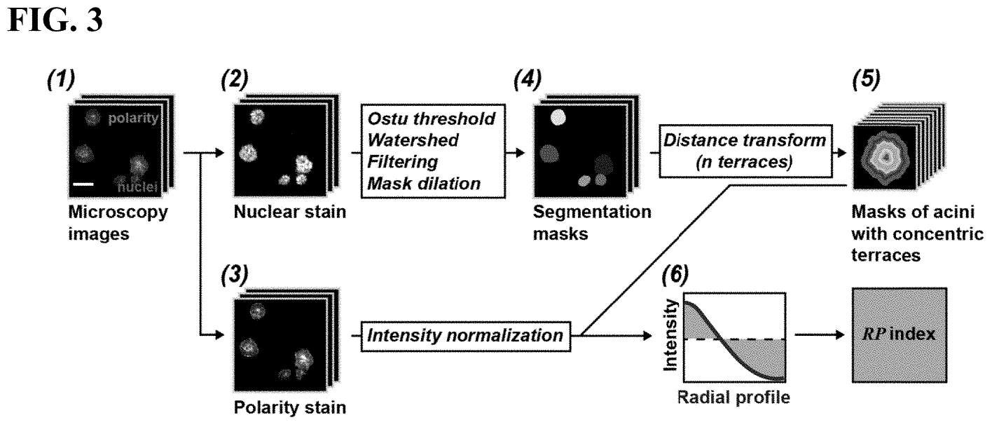

As shown in FIG. 10B, the memory M may hold various categories of software and data, such as computer readable program code PC and/or an operating system OS. The operating system OS controls operations of an electronic device 101. In particular, the operating system OS may manage the resources of the electronic device 101 and may coordinate execution of various programs by the processor P. For example, the computer readable program code PC, when executed by a processor P of the electronic device 101, may cause the processor P to perform any of the operations illustrated in the flowcharts of FIG. 2 and FIG. 3.

Example embodiments of present inventive concepts may be embodied as nodes, devices, apparatuses, and methods. Accordingly, example embodiments of present inventive concepts may be embodied in hardware and/or in software (including firmware, resident software, micro-code, etc.). Furthermore, example embodiments of present inventive concepts may take the form of a computer program product comprising a non-transitory computer-usable or computer-readable storage medium having computer-usable or computer-readable program code embodied in the medium for use by or in connection with an instruction execution system. In the context of this document, a computer-usable or computer-readable medium may be any medium that can contain, store, communicate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, or device. More specific examples (a nonexhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Example embodiments of present inventive concepts are described herein with reference to flowchart and/or block diagram illustrations. It will be understood that each block of the flowchart and/or block diagram illustrations, and combinations of blocks in the flowchart and/or block diagram illustrations, may be implemented by computer program instructions and/or hardware operations. These computer program instructions may be provided to a processor of a general purpose computer, a special purpose computer, or other programmable data processing apparatus to produce a machine, such that the instructions, which execute via the processor of the computer or other programmable data processing apparatus, create/use circuits for implementing the functions specified in the flowchart and/or block diagram block or blocks.

These computer program instructions may also be stored in a computer usable or computer-readable memory that may direct a computer or other programmable data processing apparatus to function in a particular manner, such that the instructions stored in the computer usable or computer-readable memory produce an article of manufacture including instructions that implement the functions specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide steps for implementing the functions specified in the flowchart and/or block diagram block or blocks.

Another aspect of the invention relates to kits for carrying out the methods of the invention. The kits may comprise a computer program product for carrying out the method, e.g., the RadialProfiler software disclosed herein, or provide access to the computer program product (e.g., through an online link). The kits may further comprise other tools and reagents for carrying out the methods of the invention, including without limitation, cells, matrix materials, multiwall plates, polarity marker detection agents (e.g., antibodies, affinity agents (such as an aptamer or labeled peptide), dyes, detectable labels), constructs for producing recombinant proteins, buffers, reagents, instructions, etc.

The following examples are not intended to limit the scope of the claims to the invention, but are rather intended to be exemplary of certain embodiments. Any variations in the exemplified methods that occur to the skilled artisan are intended to fall within the scope of the present invention. As will be understood by one skilled in the art, there are several embodiments and elements for each aspect of the claimed invention, and all combinations of different elements are hereby anticipated, so the specific combinations exemplified herein are not to be construed as limitations in the scope of the invention as claimed. If specific elements are removed or added to the group of elements available in a combination, then the group of elements is to be construed as having incorporated such a change.

### EXAMPLE 1

### Development of Epithelial Polarity Assay

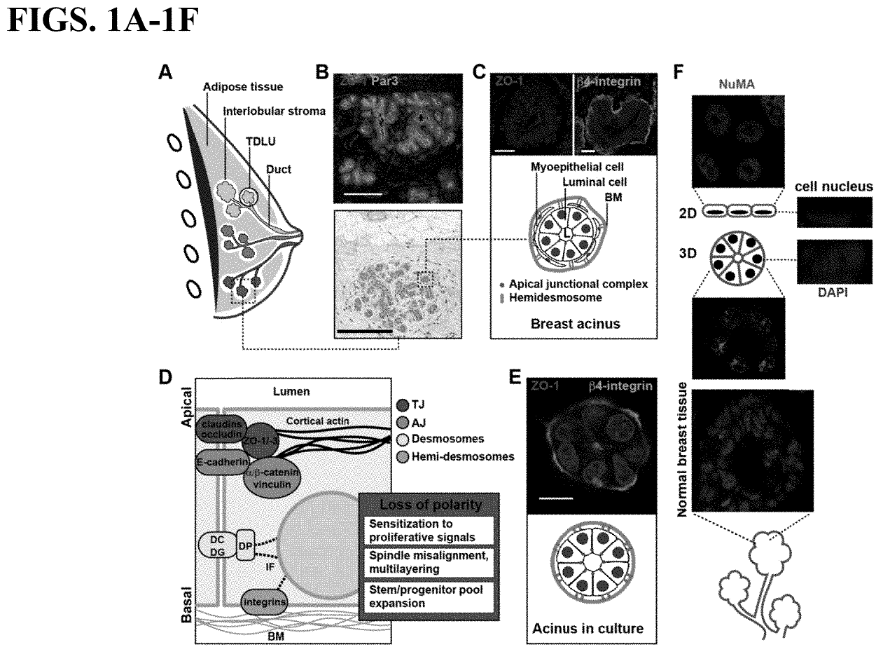

When cultured with a reconstituted basement membrane (rBM) hydrogel having physical and chemical characteristics similar to that of the basement membrane in vivo, non-neoplastic mammary epithelial cells develop into 3D structures resembling mammary gland acini (FIG. 1E). Acini cultures recapitulate important characteristics of the normal mammary gland, namely single cell-layered structures, proliferation arrest (90-95% Ki67-negative cells) and apical-basal polarity (Petersen et al., Proc. Natl. Acad. Sci. USA 89:9064 (1992); Vidi et al., Meth. Mol. Biol. 945:193 (2013)). Signaling pathways are dramatically rewired in 3D acini cultures (Furuta et al., Cold Spring Harb. Symp. Quant. Biol. 81:207 (2016)). Moreover, nuclear organization features such as gene positioning and nucleoskeletal arrangement are strikingly different in acini cultures compared to 2D monolayer cultures (Knowles et al., Proc. Natl. Acad. Sci. USA 103:4445 (2006); Meaburn et al., J. Cell Biol. 180:39 (2008)). FIG. 1F illustrates a remarkable parallel between distribution patterns of a structural nuclear protein (NuMA) in normal breast tissue and acini cultures.

Mammary epithelial cells can be cultured either embedded in or on top of rBM (Vidi et al., Meth. Mol. Biol. 945:193 (2013)). Micropatterned surfaces have also been developed as an alternative for acinar cultures (Rodriguez-Fraticelli et al., J. Cell Biol. 198:1011 (2012)). Acini cultures have the advantage of high reproducibility and manipulability. Compared to mouse models, experiments with acini cultures are cheaper, faster, raise fewer ethical concerns, and typically do not require regulatory approval.

In principle, acini models can be used for high-content analyses (HCA) at medium to high throughput—‘high-content’ referring to complex phenotypic readouts. While many screening platforms have been developed around cancer models to identify new cancer treatments, HCA protocols with normal cells for cancer prevention are scarce. Obviously, readouts based on cell killing cannot be used in the context of prevention. HCA methods to assess epithelial polarity will contribute to fill this gap.

The concept of radial profile (RP) analysis of epithelial polarity was tested. A macro script and two plugins for the open image analysis software ImageJ (imagej.nih.gov/ij/) were developed. Subsequently, computer code was translated for the MATLAB® computing platform and improvements were made. This new code is referred to as the RadialProfiler algorithm. MATLAB® is a more robust and versatile computing platform that allows important improvement to the approach. In particular, MATLAB® can easily process entire image folders in batch using looping functions. Batch processing has been included in the new MATLAB® version of the RP analysis that enables automated analyses of series of images. The code was designed to analyze two-channel fluorescence microscopy images. The first channel corresponds to DAPI staining of cell nuclei, whereas the second channel corresponds to an apical polarity marker (e.g., ZO-1, Par3, or actin) detected by immunofluorescence. The macro script identifies acini with the DAPI images and generates corresponding regions of interest (ROIs). During this segmentation step, structures that are contacting one another are separated with a watershedding approach. Filters are applied to exclude ROIs from cell structures that are out of focus (blurry sections of the images), as well as structures that do not fit size requirements. RadialProfiler uses the ROIs and analyzes the distribution of the polarity signals. Initially, two plugins were developed to compare two approaches to quantify apical polarity: (1) an analysis of the RP distribution of the signals and (2) the computation of the moment of inertia (MOI).

Non-neoplastic HMT-3522 S1 mammary epithelial cells were obtained from Dr. Mina Bissell (Lawrence Berkeley Laboratory) and propagated between passages 54 and 60 in H14 medium. Acinar differentiation was achieved by culturing the cells in 3D on top of a layer of Matrigel matrix (Corning) in chambered slides (Millipore), as described (10).

Fluorescent signals of polarity markers in 3D cell cultures were scored visually for apical localization using an Olympus IX83 epifluorescence microscope equipped with a 60× oil immersion objective (NA=1.35). At least 100 acini were scored per condition in each experiment. Alternatively, fluorescence images were taken with the CMOS camera (Hamamatsu, ORCA-Flash4.0) on this microscope, using a 10× (NA=0.3) objective. The ImageJ script was used to extract acini from DAPI images and to calculate the radial distribution of the signals. 150-450 acini were analyzed per condition.

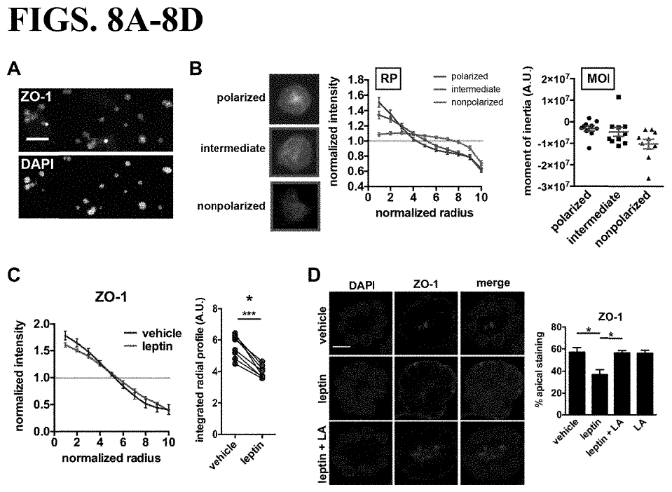

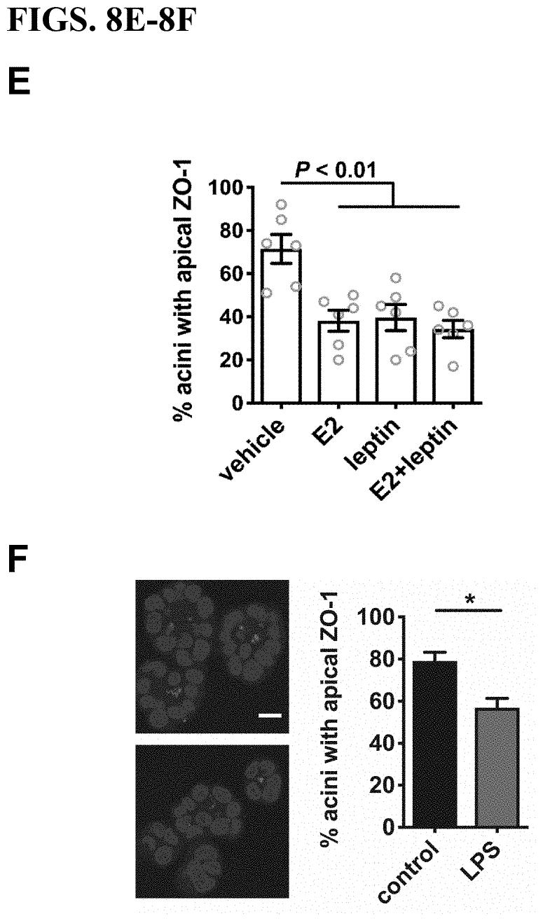

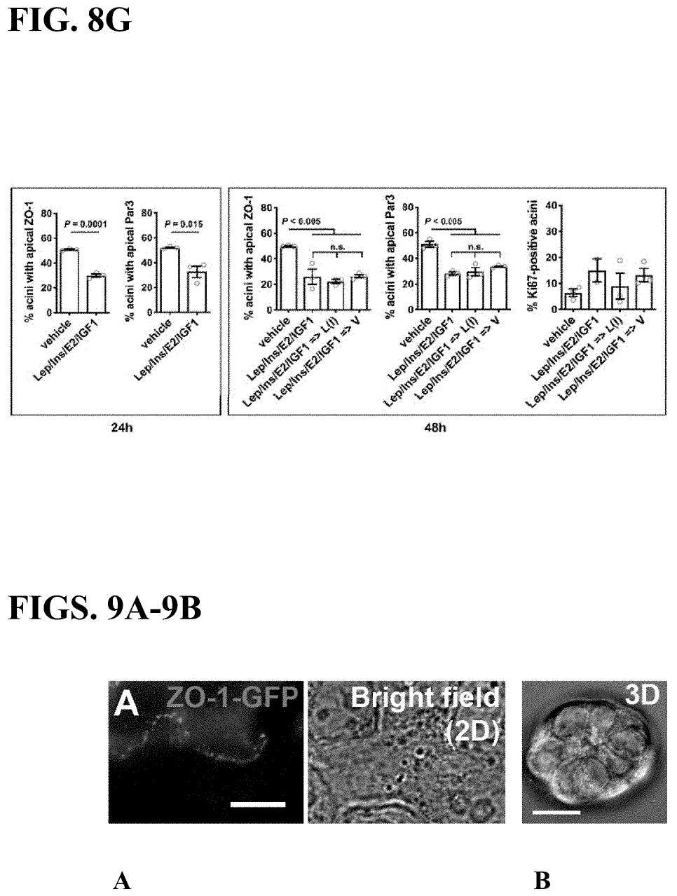

Polarized acini are characterized by apical marker signals concentrated at the center of the structures (FIGS. 8A and 8D). The first approach generates a RP plot of signal intensities from the center of the acinus to its periphery. To avoid bias from different sizes of acini and staining intensities, both the radius and the average signal intensity are normalized. The RP plots therefore represent polarity signals from center to periphery at N radial distances, with N being a user-defined number of 5-50 bins (FIG. 8B). The second approach is based on the physical concept of inertia in rotating rigid bodies (think of a ballet dancer changing the spin by extending the arms). For our analysis, we calculated ‘inverse’ MOIs as the sum of each pixel's intensity multiplied by the squared distance to the boundary of the acinus. An optimized normalizing constant was used to correct for differences in ROI sizes and intensities. With this strategy, polarized structures with signal concentrated at the center have a larger MOI (FIG. 8B).

As proof-of-concept, polarized and nonpolarized acini were visually selected from microscopy images of the ZO-1 marker and analyzed with the RP and MOI plugins. Using these images, we established that both methods quantify the apical distribution of the ZO-1 TJ marker. However, the RP method was clearly more robust.

RadialProfiler identifies and segments single or grouped acini based on a nuclear stain and separates contiguous acini with a watershed algorithm. A filtering step excludes structures smaller or larger than set values, as well as blurred, out-of-focus, acini. Regions of interest (ROIs) corresponding to individual acini are divided into concentric terraces. The boundary of the terraces roughly follow the natural boundary that was determined for each acinus. Thus, the term “concentric” as used herein is not limited to circles. The number of terraces depends on the size of the acini and the magnification used to capture images and is set by the user. The concentric terraces are then used to calculate a radial profile of polarity for each acinus. The intensity profiles are normalized to avoid influences from the staining procedure or structure sizes. In addition, the center of the acinus is defined with a radial value of zero and the periphery as a radial value of one, thereby avoiding effects linked to acini sizes. A flowchart of the analysis is shown in FIG. 2. A more detailed flowchart of the analysis is shown in FIG. 3. Steep radial profiles represent polarized structures, whereas more horizontal curves represent nonpolarized acini. Radial polarity indexes (RP) are calculated from the RP curves for direct comparisons between treatment conditions according to the equation:

RP=Σₜ₌₁^{X}|1−RPᵢ| (1)

Here, RPᵢ is the radial polarity of the i^{th} terrace. The higher the value of the RP index, the more centrally concentrated is the polarity marker. Lower RP values indicate the polarity markers are more evenly distributed radially. To distinguish between apical and basal marker distributions, positive or negative signs are assigned to RP indexes. By definition, RP indexes from curves with a negative slope (apical) are set to positive values, whereas upward RP curves (basal) yield RP indexes with positive slope values. RadialProfiler was initially implemented in ImageJ (rsbweb.nih.gov/ij/) (see Tenvooren et al., Oncogene (2019)), using an approach inspired by the Radial Profile Plot plugin from Paul Baggethun (imagej.nih.gov/ij/plugins/radial-profile.html). The algorithm was then translated for MATLAB® and the following key improvements were made: (1) addition of watershed to improve threshold-based segmentation, (2) dilation of the identified acini to account for the discrepancy between borders of nuclear-stained images as opposed to true membrane edges, (3) substitution of approximated circles with contour terracing to calculate radial profiles, and (4) addition of an exclusion criteria based on image blur to exclude out-of-focus acini. The RadialProfiler workflow is summarized below.

### Image Segmentation

Nuclear stain images are smoothened (by replacing each original pixel intensity value with the average intensity value corresponding to a 3×3 kernel size). This step reduces noise before initial segmentation, which is based on the global Otsu thresholding method. Initial segmentation usually leaves errors such as under-segmentation, where two or more adjacent acini are joined into one, larger ROI in the binary mask. To separate merged structures, the RadialProfiler algorithm applies a watershed on the binary mask obtained from Otsu thresholding. Before watershedding is applied, the borders of the identified ROIs are smoothened. To create an image for a watershed, a distance function is performed on the binary mask that reports the distance of each interior pixel to the nearest border pixel, and regional minima are found. The MATLAB® watershed function is applied on this distance image, and pixels labeled as 0 in the resulting matrix are then labeled as 0 in the binary image. Finally, acini ROIs are dilated by a certain number of pixels depending on the image magnification. This is done as the true membrane edge of the acinus lies outside of the ROI identified based on the nuclear stain.

### Filtering

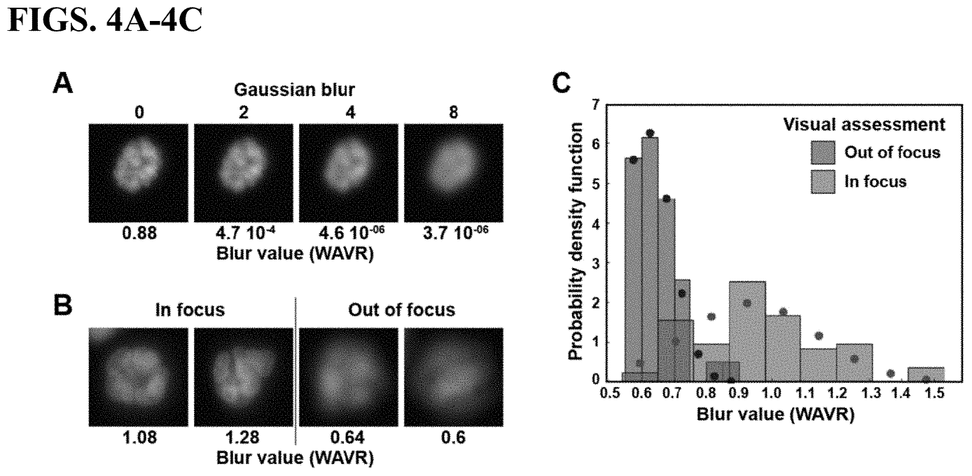

Binary masks are filtered to exclude (1) structures partially on the border of an image, (2) structures with sizes outside a specified range, and (3) structures for which the level of blur is above a user-defined cutoff. Multiple algorithms have been developed to quantify blur in an image. We compared the different approaches summarized by Pertuz et al. (Pattern Recognition 46:1415 (2013)) to determine which algorithm performed best at distinguishing blurred, out-of-focus acini based on nuclear stain images. Different levels of Gaussian blur were applied to a subset of images, creating series of images with defined levels of blurriness (FIG. 4A). Also, acini from wide field microscopy images were visually assigned to clear and blurry categories (FIG. 4B). For both approaches, it was found that a wavelet-based operator (WAVR in the Focus Measure MATLAB® function) was highly sensitive to Gaussian blurring and performed best to parse in-focus from out-of-focus acini. A plot summarizing the results is given FIG. 4C. The graph shows the WAVR probability density function for acini visually characterized as either in focus or out of focus, revealing low WAVR values for blurry structures. The WAVR values determined from a Gaussian fit were 0.61±0.08 and 0.94±0.2 (mean/SD; P<0.00001, Student's t-test) for out-of-focus and in-focus images, respectively. In this example, using a WAVR cutoff of 0.8 lead to the correct identification of 95% of acini deemed out of focus by visual evaluation, while retaining 78% of the structures visually assigned as in focus. This demonstrates that the WAVR blur value effectively distinguishes in-focus from blurry images.

### Contour Terracing

The previous algorithm (Tenvooren et al., Oncogene (2019)) discarded all acini that were not highly circular in shape because concentric circles were used to assign image pixels to the different radial zones. To lessen the amount of excluded acini and to improve precision, the current RadialProfiler algorithm defines concentric ‘terraces’ within each acinus. This step is performed using a distance transformation similar to the one used for the watershed technique. The distance transformation uses the binary mask (ROI) of an acinus. For each true pixel, the transformation returns the Euclidean distance between that pixel and the closest edge of the structure (i.e., the ROI boundary). By analogy, each acinus is treated as a “mountain”, where the edges have lowest height, and the center marks the highest elevation. Acini ROIs are converted into topographical maps with contour lines (or terraces) of equal height ranges going from the base to the peak. Having a set number of terraces (radial bin values in the software interface) is important to normalize results for comparisons between different acini of unequal sizes and between treatment conditions.

### RP Index Calculation

To calculate RP curves, the terraces defined in the previous step are imposed on the polarity images. The average pixel intensity in each terrace is calculated and divided by the average pixel intensity for the entire acinus. This normalization step yields RP curves that are not dependent on the staining efficacy (which can be uneven). The number of points for these curves is equal to the number of terraces selected. To obtain an RP index value for each acinus, each of the normalized radial intensities (RP,) are subtracted from one (the average) and the corresponding absolute values are summed—see Eq (1).

### EXAMPLE 2

### Analyses of Epithelial Polarity using RadialProfiler

The RadialProfiler algorithm was developed to analyze acini produced with non-neoplastic HMT-3522 S1 breast epithelial cells (Briand et al., In Vitro Cell Dev. Biol. 23:181 (1987)). It is expected that the radial profile method is applicable to acini produced with other normal or pre-malignant epithelial cell lines. Detailed protocols for 3D cell culture of breast acini can be found in Vidi et al., Meth. Mol. Biol. 945:193 (2013). Briefly, a thin coat of rBM (e.g., Corning Matrige™) is applied at the bottom of the culture vessel. Then, a single cell suspension (42,000 cells/cm²) is added on top of the rBM coat and is overlaid with rBM diluted in culture medium (5% final concentration) to engage the cell surface integrins that are not in contact with the rBM-coated substratum, and to promote the development of 3D structures. Different culture vessels (35 mm dishes, chambered slides, multiwell plates) are used depending on the analysis method (fixed vs. live imaging), and the throughput level (low vs. medium). For live imaging in glass-bottom dishes and plates, a thinner coat of rBM is applied to enable imaging with high numerical aperture (NA) objectives, which typically have relatively short working distances (<0.2 mm).

RadialProfiler can be applied to quantify epithelial markers detected by immunofluorescence (as described in Tenvooren et al., Oncogene (2019)), or to quantify cortical actin labeled in live acini with the SiR-actin dye (Cytoskeleton Inc.). DAPI and Hoechst are used to counterstain cell nuclei in fixed and live experiments, respectively.

For imaging, an automated IX83 microscope (Olympus) equipped with a motorized ultrasonic stage and a TruFocus Z drift compensation module was used. For RadialProfiler analyses, images are taken with either 10× (NA=0.3) or 20× (NA=0.45) air lenses, using a sCMOS camera (ORCA-Flash4.0, Hamamatsu). The RadialProfiler software was also tested with images acquired using different imaging systems, including a high content imager (Perkin Elmer Operetta CLS). RadialProfiler and the underlying approach to analyze polarity are agnostic to the imaging system. Fields of view are chosen either in an automated fashion or based on nuclear signals (DAPI or Hoechst) to avoid bias. For live cell analyses, acini are maintained at 37° C. and 5% CO₂ using a stage-top incubator (Tokai Hit). The minimal resolution needed depends on the number of radial terraces used by RadialProfiler. To improve statistical power, the number of acini in a single image needs to be maximized, which can be achieved with a low magnification objective. However, the ability to analyze the distribution of polarity markers in an acinus improves with the number of sampled image points. Lenses with higher magnification generally provide higher resolution images, with more pixels per acini, albeit with fewer acini in each field of view. In the end, the choice of magnification is directed by the need to have an individual acinus sampled at enough camera pixels to allow an accurate polarity radial profile analysis with a suitable number of terraces. It was determined that using 5-10 bins that are 2 pixels wide yields accurate measurements. This corresponds to a diameter of 20-40 pixels, which, for a circular acinus, corresponds to 316-1264 pixels. Acini are not perfect spheres; this value is therefore an estimate. This has been reinforced empirically through analysis of large data sets.

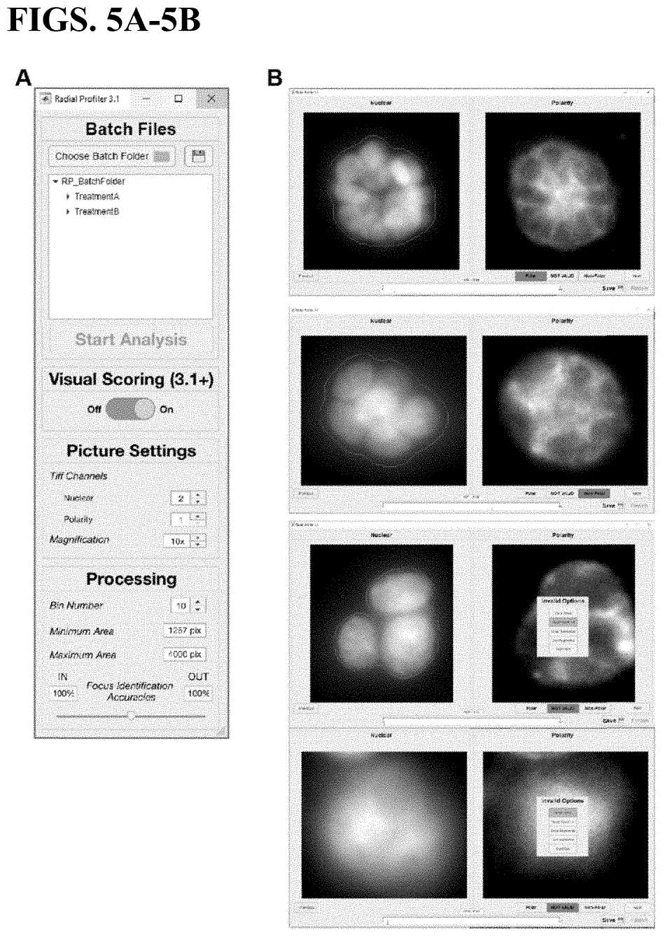

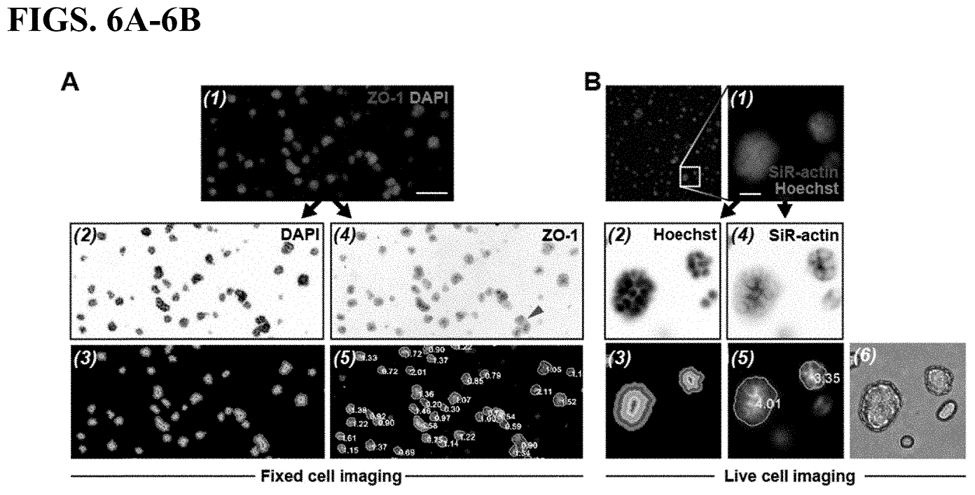

RadialProfiler operates in two modes, either supervised or unsupervised. The user chooses between these two modes with the first dialog box (FIG. 5A). The unsupervised mode runs the analysis automatically once the program parameters are set. It retrieves a table listing the normalized radial intensity values and an RP index value for each acinus. It also produces images annotated with segmentation results and RP index values (FIGS. 6A-6B). Results in the table are grouped by experimental conditions. The supervised version performs the same calculations as the unsupervised version but also includes a graphical user interface (FIG. 5B), allowing the investigator to visually score polarity and assess the quality of the acini identification steps (segmentation efficacy, blurriness, etc.). Individual acini are presented to the user in a randomized order and without providing any treatment information, which enables blind scoring. After completion of visual scoring, a table with RP index values and user scores is produced. Representative results are shown in FIGS. 6A-6B.

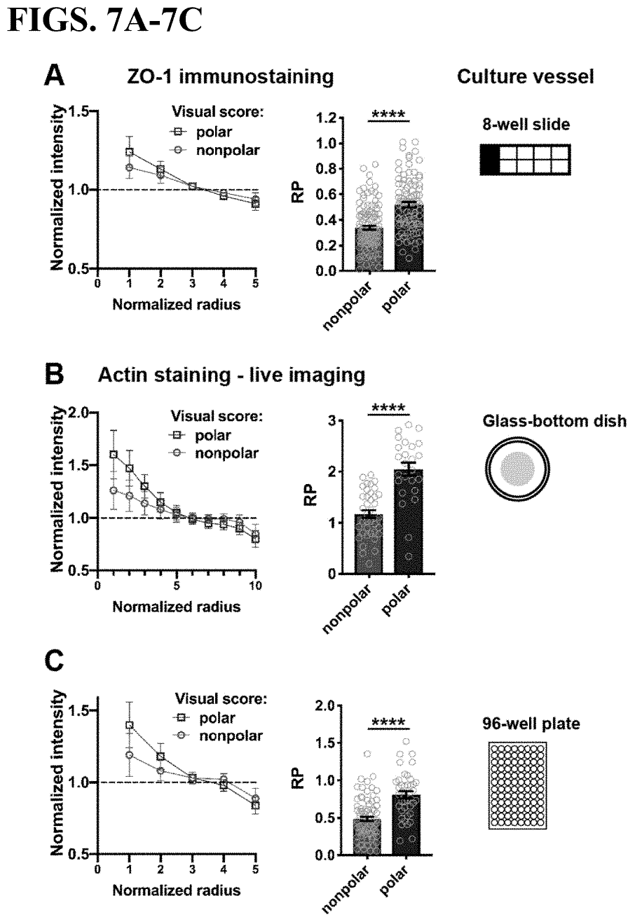

In conclusion, a method to quantify epithelial polarity in breast acini organoid cultures was developed. The method is based on radial marker profiling and results in a single polarity index to assess establishment or breakdown of apical-basal polarity in populations of acini. This method should be applicable to a wide variety of cell types and treatment conditions. The software interface is user-friendly and circumvents the need to use command lines in MATLAB®. RadialProfiler is therefore accessible to biologists and health scientists with minimal knowledge of the computing platform. Importantly, the RP index produced by the software successfully distinguishes between non-polar and polar acini, as demonstrated in the analyses presented in FIGS. 7A-7C. Similar results were obtained using different imaging platforms.

It is anticipated that this assay will fill unmet needs in primary prevention of breast cancer and other carcinomas, with applications including (1) chemoprevention drug screening (2) toxicology assessment of suspected carcinogens and pharmacological lead compounds, and (3) personalized cancer risk diagnosis. High content screening methods for cancer prevention are scarce. Since loss of apical-basal polarity is an early step enabling the initiation of carcinoma, an assay of epithelial polarity may be used to screen for chemoprevention drugs or natural compounds preventing polarity loss or restoring polarity. The RP assay may also be implemented to weed out drug candidates with toxic effects on the epithelial architecture before testing in mouse models. Indeed, the vast majority of hit compounds in drug discovery pipelines fail the transition from the initial screen to animal models. Relevant in vitro assays, such as the RP assessment, may be used to rapidly and cheaply screen for toxic effects on normal cells, thereby reducing the need for animal research, which is expensive and raises ethical concerns. More broadly, assays with non-neoplastic cell organoids can be used to assess suspected carcinogens (Meng, Expert Opin. Drug Metab. Toxicol. 6:733 (2010); Grabinger et al., Cell Death Dis. 5:e1228 (2014); Rocco et al., Toxicol. Sci. 164:592 (2018)).

Current breast cancer risk assessment methods provide population-based estimates of risk rather than personalized risk assessment. Genetic testing can identify mutations associated with cancer risk (e.g., BRCA1/2 for breast cancer), yet only a small fraction of malignancies (about 5% for breast cancer) have a known genetic origin. Cell-phenotypical assays, including epithelial polarity readouts, may be used to rapidly assess personalized breast cancer risk, for example for women participating in lifestyle interventions. In these cases, acini cultures and RP analyses may serve as biomarkers for integrative assessment of improvements in metabolic risk factors.

### EXAMPLE 3

### Application of Epithelial Polarity Assay to Breast Cancer

As part of a research project on obesity and breast cancer risk, it was discovered that elevated levels of the adipokine leptin—a key link between obesity and cancer—leads to loss of apical polarity, as measured with conventional methods (Tenvooren et al., Oncogene (2019)). These observations, together with a large body of literature showing the critical importance of apical-basal polarity disruption for cancer initiation, was the trigger to develop a platform to efficaciously quantify polarity in a physiologically relevant cell culture model—the RP approach.

To derive summary values from RP plots, an approximate integrated deviation of the RP curves (defined as Σ (|x-1|) was computed for the N bins, with x the normalized signal intensity). In the latest version a sign was set for the RP index (positive for apical signals and negative for basal signals). This approach was applied to images taken from experiments where acini were treated with either vehicle or leptin. Both the averaged RP curves and integrated values were distinct for the two treatments (FIG. 8C), validating visual scoring at the microscope (FIG. 8D).

Another experiment was performed to study the effect of elevated estrogens (E2) alone or in combination with leptin on breast acini. The results are shown in FIG. 8E.

A similar experiment was performed to study the effect of lipopolysaccharide (LPS) on acini. The results are shown in FIG. 8F. Exposure to LPS resulted in a decrease in the polarity score.

The effect of transient exposure of breast acini to elevated growth factors was measured. Acini were cultured for 10 days, treated for 24 hours with elevated levels of leptin, insulin, estrogens, and IGF-1, then recovered for 24 hours. Polarity was measured after treatment and after recovery. The results are shown in FIG. 8G.

### EXAMPLE 4

### Live Cell Imaging Assays

Immunostaining techniques have certain limitations, including (1) time consumption, (2) costs of reagents, and (3) poor time resolution. An alternative approach has been developed based on live cell imaging. Immunostaining for polarity markers is replaced by expression of polarity marker proteins tagged with a fluorescent protein (e.g., GFP-ZO-1). A mammary epithelial cell line stably expressing GFP-ZO-1 has been generated and characterized (FIGS. 9A-9B). The cell line retains apical-basal polarity (which is disturbed by leptin treatment) and expresses the recombinant marker at physiological levels. As a more flexible alternative to genetically encoded fluorescent markers of polarity, a nontoxic actin dye compatible with live cell imaging (SiR-actin) has been used. Cells are stained for 1 h with a mix of SiR-actin and Hoechst (DNA dye) before imaging. Similar results were obtained with this approach compared to the fixed, immunostaining approach described in EXAMPLE 1. Specifically, leptin treatment (as in EXAMPLE 3), led to the loss of apical SiR-actin signal in live S1 cell acini.

All publications, patents, and patent applications are herein incorporated by reference to the same extent as if each individual publication, patent, or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

1. A method for quantitating epithelial cell apical-basal polarity in a three-dimensional multi-cell structure, comprising:
a) detecting a polarity marker in the three-dimensional multi-cell structure;
b) capturing an image of the three-dimensional multi-cell structure in which the polarity marker has been detected;
c) segmenting the image into multicellular units having a level of apical-basal polarity;
d) measuring intensity of the polarity marker across a multicellular unit; and
e) calculating a polarity score for the multicellular unit based on the intensity distribution of the polarity marker;
thereby quantitating epithelial cell polarity.

2. The method of claim 1, wherein the polarity of the multicellular unit is radial polarity.

3. The method of claim 1, wherein two or more images of the labeled three-dimensional multi-cell, structure are captured.

4-5. (canceled)

6. The method of claim 1, wherein the three-dimensional multi-cell structure comprises at least a portion of an exocrine gland (acinus) or a cell culture equivalent of an exocrine gland.

7-8. (canceled)

9. The method of claim 1, wherein two or more polarity markers are detected in the three-dimensional multi-cell structure.

10. The method of claim 1, wherein the polarity marker is a protein, lipid, organelle, or other molecule or structure that is present in a cell in a polarized manner.

11-15. (canceled)

16. The method of claim 10, wherein the protein is afadin, claudin-1, ZO-1, ZO-2, ZO-3, Par3, a connexin, E-cadherin, β-catenin, GM130, β4-integrin, α6-integrin, or collagen IV.

17. The method of claim 1, wherein the polarity marker is a cytoskeleton element that is present in a cell in a polarized manner.

18. The method of claim 17, wherein the cytoskeleton element is cortical actin filament.

19. The method of claim 1, wherein detecting the polarity marker comprises labeling the polarity marker with an antibody, an affinity agent, or a dye.

20. The method of claim 1, wherein the polarity marker is a protein recombinantly linked to a detectable label.

21. The method of claim 1, wherein step a) further comprises detecting at least one other portion of the cells.

22. The method of claim 21, wherein the at least one other portion of the cells is the nucleus.

23. The method of claim 1, wherein step c) comprises identifying the centroid of the multicellular unit and the boundary of the multicellular unit.

24. The method of claim 1, wherein step c) comprises identifying the boundary of the multicellular unit and then determining the most interior point(s) from the boundary.

25. The method of claim 24, wherein the interior region of the multicellular unit is divided into zones or terraces whose pair of boundaries roughly mimic the shape of the outer boundary of the multicellular unit.

26. The method of claim 25, wherein the average intensity of the pixels in each terrace is determined, producing a set of points forming a radial polarity curve of average terrace (or bin) intensity vs. bin number, where the most interior terrace or bin is the first bin, and as one progresses radially out the bins are incremented by 1 for each new terrace that is encountered.

27. The method of claim 26, wherein the radial polarity curve is normalized by dividing each average bin intensity by the average intensity of the multicellular unit.

28. The method of claim 27, wherein the radial polarity curve is used to determine the polarity score.

29. The method of claim 28, wherein the polarity score consists of two numbers, one corresponding to the integral or sum of the absolute values of 1 minus the average intensity of each bin and the sign of the slope of a linear fit to the normalized radial polarity curve.

30. The method of claim 1, wherein calculating. a polarity score comprises dividing the area of the multicellular unit into 2 or more bins based on distance from a point in the image and measuring the intensity of the polarity marker in each bin.

31. The method of claim 30, wherein distances are calculated from the center of mass of the multicellular unit.

32. (canceled)

33. The method of claim 30, wherein the intensity in each bin is normalized to the average intensity in the multicellular unit.

34. The method of claim 33, wherein the normalized intensity is plotted versus distance from the point in the image to generate a polarity curve.

35. The method of claim 34, wherein the polarity curve is integrated to provide a single value for the polarity score.

36. The method of claim 1, wherein a polarity score is calculated for at least two multicellular units and an average polarity score is determined.

37-49. (canceled)
